(19) United States
(12) Patent Application Publication
(10) Pub. No.: US 2004/0048262 A1
(43) Pub. Date: Mar. 11, 2004

(54) Methods for determining the bilogical effects of compounds on gene expression

(76) Inventors: Christopher Adams, San Diego, CA (US); Paul Labhart, San Diego, CA (US); Mary E. Harper, San Diego, CA (US)

Correspondence Address: Hale & Dorr, 300 Park Avenue, New York, NY, 10022, US

(21) Appl. No.: 10/250,802

(22) PCT Filed: Nov. 9, 2001
(86) PCT No.: PCT/US01/46927

(51) Int. Cl. C12Q 1/68

(52) U.S. Cl. 435/6

(57) ABSTRACT

Methods for determining one or more biological effects of a compound on gene expression are described. These methods involve obtaining a nuclear extract from cells exposed to a compound and then combining the nuclear extract with a nucleic acid, or library of nucleic acids, containing-one or more regulatory elements under conditions that allow formation of cis/trans complexes between the regulatory elements and components (e.g., DNA binding proteins) of the nuclear extract. The complexes so formed are then compared with complexes formed using nuclear extracts obtained the same but untreaed cells and the compound. Differences between the comlexes formed as a result of exposure of teh cells to the compound can then be assessed. The methods of th einvenion are preferably carried out in a high throughput format, and are useful, for example, to assess efficacy, toxicity, and mechanism of action of a compound. Accordingly, the invention will be useful in developing new drugs, and in better understanding and improving compounds already in use or under development.

### FIELD OF THE INVENTION

This invention in general relates to methods for determining the biological effect(s) of a compound. More specifically, this invention discloses methods of examining the effect(s) of compounds by measuring changes in gene expression. Accordingly, the invention can be used to assess compound efficacy, toxicity, mechanism of action, etc. As such, it will have widespread use, for example, in developing novel pharmaceutical compounds as well as in testing effects on gene expression of these and known compounds.

### BACKGROUND OF THE INVENTION

The following description of the background of the invention is provided to aid in understanding the invention, but is not admitted to be or to describe prior art to the invention.

The regulation of gene expression is critical to the growth, development, proliferation, and maintenance of all living cells and organisms. In most cases, the positive or negative regulation of genes is under the control of signal transduction cascades which transmit information from the cell surface to the nucleus. Signal transduction cascades are generally triggered by ligands which may be small molecules, soluble peptides, extracellular matrix, adhesion proteins projected from the exterior surface of neighboring or migrating cells, and even metabolic intermediates. In most cases, ligands interact with a membrane bound, or sometimes soluble intracellular, receptor, thus triggering a cascade of events that ultimately either stimulates or inhibits the expression of one or more genes. Such reprogramming of gene expression leads to a, hopefully appropriate, cellular response to the stimuli. Based on current understanding, almost all such signals converge and mediate their function through activators and/or repressors of RNA transcription, including those that act indirectly by effecting chromatin structure.

Because of the importance of gene expression to living cells, manipulating the process is of extreme interest. Compounds that fundamentally alter the activity of the transcriptional machinery itself, for example, by inhibiting the elongation process, would be potent transcriptional modulators, but would probably not be gene-specific, and it is gene-specific regulation that is the goal of many development programs. One approach to gene-specific transcriptional regulation has been to develop molecules that block activator-DNA or repressor-DNA interactions and thereby regulate transcription artificially. Several approaches in this vein are being investigated, including the use of protein nucleic acid (PNAs; oligomers that contain the standard purine and pyramidine bases of an oligonucleotide but contain a simple amide-based backbone as opposed to the sugar-phosphate backbone found in nucleic acids; Nielsen, P. E. (1997) Chem.Eur. J. 3, 505-508; Footer, M., et al. (1996) Biochemistry 35, 10673-10679), oligonucleotides that are capable of promoting “triple helix” formation, and a class of sequence-specific molecules known as “polyamides” (see, e.g., Dervan, et al., Curr. Opin. Chem. Biol. (1999), vol. 3: 688-693; Bremer, R. E., Baird, E. E. & Dervan, P. B. (1998) Chem. Biol. 5, 119-133).

In addition to developing molecules that interfere with the association of activators and repressors with their cognate target sequences in double-stranded DNA, another approach involves compounds that modulate interactions between proteins involved in the regulation of transcription or chromatin structure. To date, efforts in this area have involved cell-based genetic approaches. For example, the socalled “two-hybrid assay” (Fields, S. & Song, O. K. (1989) Nature 340, 245-246) is based on the observation that in many promoter contexts, the DNA binding and activation domains of an activator protein function more or less independently of one another (Vashee, S. & Kodadek, T. (1995), Proc. Natl Acad. Sci. USA 92, 10683-10687; Vashee, S. et al. (1998) Curr. Biol. 8, 452-458), but require functional association in the proximity of the promoter. For instance, if the activation and DNA binding domains of the yeast Gal4 protein are severed and expressed in a yeast strain deleted for wild-type GAL4, no transcription of genes (e.g., a reporter gene) under the control of GAL4 promoter occurs. However, when genes encoding two other proteins that interact with one another are fused to the DNAs encoding the severed GAL4 domains, activator activity may be reconstituted and the target gene can be transcribed (Phizicky, E. M. & Fields, S. (1995) Microb. Rev. 59, 94-123). Other similar systems, each of which requires the intracellular expression of chimeric gene constructs, have been reported. See, e.g., Vidal M., et al. (1996), Proc. Natl Acad. Sci. USA 93, 10321-10326; Leanna, C. A. & Hannink, M. (1996), Nucleic Acids Res. 24, 3341-3347; Huang, J. & Schreiber, S. L. (1997), Proc. Natl Acad. Sci. USA 94, 13396-13401; Hu, J., et al. (1990), Science 250, 1400-1403.

Despite these approaches, however, at present there exists a need to develop improved methods for determining the biological effect(s) of a compound on gene expression and to address this need the instant invention is provided.

### SUMMARY OF THE INVENTION

It is an embodiment of this invention to provide methods for determining the biological effect(s) of one or more compounds on the expression of genes in human, animal, multi- and single celled organisms and viruses. Thus, a first aspect of the invention concerns methods for determining a biological effect (e.g., efficacy, toxicity, resistance, and mechanism of action) of one or more compounds on such gene expression. By “biological effect” is meant the influencing of the metabolism or biochemistry of a cell. With respect to the current invention, such effect preferably is one the influences either directly or indirectly expression mechanisms, pathways, etc. of a cells gene pool. By “efficacy” is meant the ability of a compound to induce changes in transcription factor binding activities consistent with efficacy for that particular compound. By “toxicity” is meant changes in transcription factor binding activities consistent with toxic events in cells. By “resistance” is meant the ability of a compound to cause changes in transcription factor binding activities consistent with the cell demonstrating resistance to the particular compound.

The methods of the invention comprise obtaining a nuclear extract from cells that prior to obtaining the nuclear extract were exposed to a compound of interest, and combining the nuclear extract with a nucleic acid containing a cis-binding site (also sometimes referred to as a regulatory element or cis element) under conditions that allow formation of transcription factor/cis site complexes, such complexes being well understood by those of ordinary skill in the art. Preferably, the nucleic acid containing such cis-binding site is a library or plurality of nucleic acids each comprising one more, and preferably different binding sites. The transcription factor/cis complexes so formed are then compared with the transcription factor/cis complexes formed using a like nucleic acid (or library of nucleic acids) to form complexes with a control nuclear extract obtained from cells that had not been exposed to the compound.

By “cis-binding” is meant any cis element of defined nucleotide sequence that can be identified in a nucleic acid molecule and which associates with an endogenous DNA-binding compound of the transcriptional machinery. Such elements include promoters and enhancers. A “promoter” is the minimum sequence necessary to initiate transcription of a target gene by an RNA polymerase, for example, in eukaryotic cells, RNA polymerase I (which transcribes ribosomal RNA (rRNA) in eukaryotic cells), RNA polymerase II (which transcribes messenger RNA (mRNA) in eukaryotic cells), and RNA polyrmerase III (which tnanscribes transfer RNA (tRNA) in eukaryotic cells). An “enhancer” is a cis-acting sequence that increases the utilization of a eukaryotic promoter. Preferred cis elements that are included in an oligonucleotide are those that occur endogenously in association with the gene whose transcription is to be regulated. As such, promoters from which transcription can be initiated can be targeted.

As used herein, “regulate” or “modulate” refers to an ability to alter the level of expression of a particular gene above (i.e., up-regulate or activate) or below (i.e., down-regulate or repress) the basal level of expression that would occur in the particular system (for example, an in vitro transcription system or a cell) in the absence of a compound of interest under the same conditions. A compound that activates transcription is referred to herein as an “activation moiety” or “activator,” whereas a compound that represses transcription is referred to as a “repressor moiety” or “repressor”.

Certain preferred embodiments of the methods of the invention use nucleic acids that are comprised of two completely or partially complementary oligonucleotides that completely or partially overlap with one another. Preferably, an oligonucleotide used in the practice of a method according to the invention will contain at least one regulatory element. In certain embodiments, the oligonucleotides comprise a plurality of, i.e., 2, 3, 4, 5, 6, 7, 8, 9, 10, or more, regulatory elements. Such an oligonucleotide may comprise a defined nucleotide sequence. Certain preferred oligonucleotides comprise nucleotide sequences that are representative of a genome. Other preferred oligonucleotides comprise nucleotide sequences actually found in genomic DNA. Alternatively, the nucleotide sequence may be random. A “defined nucleotide sequence” refers to a specific sequence of nucleotides, and is typically represented in the 5′ to 3′ direction using standard single letter notation. Deoxynucleotides, or nucleotides, are referred to according to standard abbreviations: “A”, deoxyadenylate; “C”, deoxycytidylate; “G”, deoxyguanylate; “T”, deoxythymidylate; “M”, A or C; “R”, A or G; “W”, A or T; “S”, C or G; “Y”, C or T; “K”, G or T; and “N”, A, C, T or G. It is understood by those skilled in the art that T bases in DNA molecules are replaced by uridine (“U” bases) in the corresponding RNA molecules. It will be appreciated that an oligonucleotide having a defined nucleotide sequence may include a different nucleotide at the same position, i.e., is degenerate at that position, with respect to one or more positions in the particular sequence. Degenerate bases may be represented by any suitable nomenclature, for example, that which is described in World Intellectual Property Organization Standard ST.25 (1998), Appendix 2. Random oligonucleotides may also comprise nucleotide sequences representative of a genome. For example, an oligonucleotide may comprise the same bias for nucleotide representation as a particular genome. Oligonucleotides may also contain modified nucleotides, for example, methylated nucleotides, as well as, or alternatively, nucleotide analogs and derivatives.

In some preferred embodiments, the methods of the invention employ libraries of different complementary oligonucleotide species. Preferably, the members of the library contain various differing cis-binding sites. When one or more of the double-stranded oligonucleotide species present in a library contain more than one cis-binding sites, it is preferred that they be different cis-binding sites, although the invention does contemplate double-stranded oligonucleotides that contain multimers of the same, or several different cis-binding sites.

Other embodiments of the invention concern the use of oligonucleotides that comprise a first amplification primer site upstream of the cis-binding site(s) and a second amplification primer site downstream of the cis-binding site(s). The primer sites can be used to amplify the regions disposed therebetween by a suitable amplification process, for example, PCR, strand displacement amplification, and trancription mediated amplification.

In some embodiments of the invention, the nucleic acid molecules are attached to or otherwise localized at a solid support. Preferably, there are a plurality of different nucleic acid species attached to different locations on the solid support.

In another embodiment, nuclear extracts are used in the methods of the invention and can be obtained from any of a variety of cells. A “nuclear extract” refers to a preparation obtained from cell nuclei. Preferably, such preparation contains proteins found in the nucleus that retain their biological activities. Preferably, a nuclear extract will be substantially free from naturally occurring lipid and nucleic acid components. Nuclear extracts may be derived from any prokaryotic or eukaryotic plant or animal cell, including cells grown in vitro (including cells cultured ex vivo) or in vivo. In certain preferred embodiments, the cells are vertebrate cells, particularly mammalian cells such as canine, equine, feline, murine, ovine, porcine, and primate cells. Particularly preferred are human cells. Other preferred vertebrate cells include avian and fish cells. Other preferred cells include pathogen cells, for example, yeast and bacterial cells. In addition, cells infected by a pathogen, for example, viruses or bacteria, can also be used in the practice of the invention. Other embodiments of the invention concern diseased cells and normal cells. Representative examples of diseased cells include cancer cells, virally infected cells, abnormal T cells, and abnormal neuronal cells.

In another embodiment, compounds are screened according to the instant methods, such compounds including natural and synthetic compounds of unknown or known activity. Natural compounds are derived from natural products, and include products present in extracts and in other less purified forms. By “synthetic” is meant any compound not found in nature, i e., in a wild-type animal, plant, or virus. Synthetic compounds include non-naturally occurring analogs, derivatives, and other modifications of natural compounds. Synthetic compounds frequently are derived by combinatorial methods. Indeed, an initial lead compound identified according to the instant methods may be further modified in a program of medicinal chemistry to optimize its desired properties, and/or to minimize its deleterious effects or other undesirable attributes.

Synthetic compounds may be synthesized by solution or solid phase methods. Two or more moieties may also be synthesized together. Compounds useful in the practice of the invention can be in unpurified, substantially purified, and purified forms. The compounds can be present with any additional component(s) such as a solvent, reactant, or by-product that is present during compound synthesis or purification, and any additional component(s) that is present during the use or manufacture of a compound or that is added during formulation or compounding of a compound.

In general, a regulatory compound useful in the practice of the invention is any compound that can positively or negatively effect, by either a direct mechanism (i.e., by direct interaction with one or more components of the transcription complex) or an indirect mechanism (i e., by (i) direct interaction with a repressor protein or (ii) direct interaction with a protein involved in chromatin or nucleosome structure), transcription of a target gene. Further compounds capable of being screened include any compound having an ultimate effect on the gene regulatory profile of a cell whether the compound acts directly or indirectly on the metabolic pathways involved in gene regulation and transcription factor/cis site complex formation.

In further embodiments, compounds may not have a direct effect on gene regulation, but may directly affect one of the many other processes in a cell. Examples include binding to one or more of the numerous cell components other than those involved in gene transcription such as those affecting negatively or positively processes such as cell metabolism, signal transduction, apoptosis, protein secretion, RNA translation, ion transport, respiration, lysosomal makeup, nuclear trafficking, cell cycling, and the myriad of other processes associated with a normal (or diseased) physiologic state of a cell. In this aspect, the methods of the invention examine the effect a compound may have on a cell that ultimately affects changes in the expression of certain genes.

Representative embodiments of compounds include peptides, polypeptides (including naturally occurring or synthetic mutant polypeptides), nucleic acids, lipids, carbohydrates, small organic molecules, and any combination thereof A “peptide” is a polymer (i.e., a linear chain of two or more identical or non-identical subunits joined by covalent bonds) made up of naturally occurring or synthetic D- or L-, or D- and L-, amino acids joined by peptide bonds. Generally, peptides contain at least two amino acid residues (ie., the molecule resulting from the formation of a peptide bond between two amino acids, or between an amino acid residue and another amino acid) but fewer than about 50 amino acid residues. A “polypeptide” is also a polymer of amino acid residues linked by peptide bonds, but typically contains at least about 50 amimo acid residues. Thus, herein, “peptide” is used to refer to a regulatory moiety that is less than about 50 amino acid residues in length, and “polypeptide” refers to larger polymers of amino acid residues linked by peptide bonds. A “nucleic acid” is any polymer of nucleotides, be they natural (e.g., A, G, C, or T) or synthetic, and whether linked by phosphodiester or other chemical linkages. A “lipid” is a substantially water-insoluble molecule that contains as a major constituent an aliphatic hydrocarbon. Lipids include fatty acids, neutral fats, waxes, and steroids. The hydrocarbon portions of the molecule may be of any length, may be saturated or unsaturated, and may be straight- or branched-chain. “Carbohydrate” refers to any aldehyde or ketone derivative of a polyhydric alcohol, and includes starches, sugars, celluloses, and gums. Particularly preferred regulatory compounds are small organic molecules (i.e., a water soluble organic molecule having a molecule weight of less than about 5,000 daltons, preferably less than about 2,500 daltons, more preferably less than about 1,500 daltons, and most preferably less than about 1,000 daltons).

Preferably, the methods of the invention are performed in vitro, preferably in a high throughput format, meaning that more than about 10, preferably, more than about 100, 1,000, or 10,000 compounds are screened at once. As will be appreciated, compounds may also be pooled. Alternatively, a variety of parameters may be screened, for example, different compound concentrations, nuclear extracts generated after different times following compound addition, etc. In certain embodiments, the regulatable gene is a marker gene, such as a gene encoding a luciferase or green fluorescent protein.

In further preferred embodiments of the invention, an expression profile is performed after it is determined which cis-binding sites were bound or unbound by a protein in response to exposure of a cell population (in vivo or in vitro) to one or more compounds. In certain preferred embodiments, the expression profile is determined by performig RNA profiles of the cells cultured or grown in the presence or absence of the compound. An RNA profile may be performed by any suitable method, for example, by nucleic acid hybridization. Preferred hybridization techniques include the use of a nucleic acid array that comprises probes, or hybridization tags, for the subset of genes expressed in the cells or, preferably, for the genes known to be functionally associated with the particular cis-binding sites determined to change as a result of compound treatment. Alternative expression profile embodiments are based on the detection of proteins expressed in the treated cells from genes known to be functionally associated with a particular cis-binding site(s).

Another aspect of the invention concerns a method for determining a biological effect of a compound of interest whereby a nuclear extract from cells exposed to the compound is prepared and then reacted with a solid support to which is attached a nucleic acid molecule containing a cis-binding site for specific interaction with a protein associated with regulating transcription of one or more genes under conditions that allow formation of a transcription factor/cis-binding site complex. The complexes formed as a result of the foregoing reaction are then compared with the complexes that are formed using a control nuclear extract obtained from cells not exposed to the compound.

Another method for determining a biological effect of a compound of interest involves taking a nuclear extract from cells exposed to the compound and reacting that nuclear extract with a DNA library to form transcription factor/cis site complexes. The complexes are then characterized by reacting them with a solid support to which is attached an antibody specific for a protein associated with said complex. Preferably, the analysis would involve analysis of many of the transcription factors likely to be active in the particular cells used for testing the compound. The results obtained as a result of the foregoing reaction are then compared with the results obtained using a control nuclear extract obtained from cells not exposed to the compound.

The above summary of the invention is not limiting and other features and advantages of the invention will be apparent from the following detailed description, as well as from the appended claims abstract.

### BRIEF DESCRIPTION OF THE DRAWINGS

FIG. 1 is a bar graph showing the effects of three different drug compounds, doxorubicin, taxol, and tamoxifen, on the levels of DNA-binding activities for a limited set of transcription factors (names listed on the X-axis) in MCF7 cells. The level of DNA binding activity for the individual transcription factors is depicted as a percentage of the total number of DNA fragments sequenced from the “bound” fraction of the DNA library used in the binding reactions and containing the cognate site for that particular protein (shown as a numerical value on the Y-axis). For example, a little over 2% of the sequenced fragments from the protein “bound” population for the tamoxifen, taxol, and doxorubicin treated cells contain an AHRARNT binding site compared to less than 0.5% of the sequenced fragments for the untreated (control) cells. This would indicate that the binding activity of this particular transcription factor was induced by all three drug treatments, suggesting that expression of genes under the control of this factor would be altered in drug treated cells versus the control cells. If these genes were known to be involved in apoptosis, for instance, then one could infer that tamoxifen, taxol, and doxorubicin treatment has some effect on cell death. The level of binding activities for all the proteins detected as a result of their cognate binding sites being found in the “bound” fraction of the DNA library constitutes the transcription factor activity profile resulting from the specific drug treatment. This profile is analogous to a diagnostic fingerprint indicating the effects of any specific drug compound on the overall activities of all transcription factors in the cells being treated. Differences in individual transcription factor DNA-binding activities can be directly correlated to changes in the expression of genes being regulated, either directly or indirectly, by that protein factor.

FIG. 2 is a bar graph showing the effects of nerve growth factor (NGF) treatment on transcription factor DNA-binding activities in PC12 cells. The identity of the specific transcription factors whose binding activity is being detected are listed on the X-axis. The level of DNA binding activity for each individual transcription factor is depicted as a percentage of the total number of DNA fragments sequenced from the “bound” fraction of the DNA library used in the binding reactions and containing the cognate site for that particular protein (shown as a numerical value on the Y-axis). The level of binding activities for all the proteins detected as a result of their cognate binding sites being found in the “bound” fraction of the DNA library constitutes the transcription factor activity profiles for PC12 cells and PC12 cells following NGF treatment. The profiles generated provide a useful indicator of the mechanism of action of the compound being used for the cell treatrment. For example, if the mechanism of action of NGF was to increase the expression of a particular cell surface receptor, then one would expect to see altered levels of binding activities for those transcription factors known to regulate the expression of the gene that encodes that particular receptor in the NGF-treated sample. If one did indeed see altered binding activity levels of factors known to regulate a particular receptor gene or set of genes, then one could immediately assay for those changes in gene expression as a result of inference from the transcription factor activity profile.

### DETAILED DESCRIPTION OF THE INVENTION

The present invention concerns novel, useful, and non-obvious methods that allow a biological effect of a compound (e.g., efficacy, resistance, mechanism of action, and toxicity) to be determined. Before describing these methods in detail, a brief overview of gene expression is provided.

Overview of Gene Expression

The human genome is believed to contain between 50,000 to 100,000 genes, each of which encodes at least one protein or RNA molecule. Each of these genes comprises or produces at least three types of nucleic acid molecules having different molecular characteristics. A gene per se is a double-stranded DNA (dsDNA) molecule that includes elements that control and act as a template for the production of RNA molecules from one of the two strands of the dsDNA; the RNA molecules produced during transcription have the same nucleotide sequence as the template strand of the dsDNA but are chemically distinct from DNA molecules; and chemically distinct RNA:DNA hybrid nucleic acid molecules exist, albeit temporarily, as RNA is synthesized from its DNA template. RNA hybrid molecules are also produced during the replication of retroviruses due to the action of reverse transcriptase. With rare exceptions, the nucleus of each cell of a multicellular organism contains a full genome complement. However, the full complement of genes is not expressed in any one cell at any one time. This difference in gene expression between cells gives rises to the observed differences in cells (e.g., nerve cells are different from muscle cells, normal cells are different from diseased cells, etc.) due to the expression of different genes. Thus, it is the coordinated pattern of differential expression of only a subset of genes in the nucleus of a given cell type that distinguishes cells of that type (e.g., nerve, muscle, bone, connective tissue, vascular tissue, skin, etc.) from other types of cells.

The major players in the regulation of gene expression within the nucleus are: the genes and their regulatory sequences which are complexed with structural proteins (e.g., histones) in chromatin; chromatin remodeling activities which allow access to a gene and its regulatory regions; regulatory proteins which instruct the transcription machinery to express (or, as in the case of repressors, prevent the expression of) the relevant genes; and the RNA-synthesizing machinery which decodes the genes. A host of other activities play a role in this process, for instance, those that facilitate elongation of paused transcripts, or those that lead to the processing of nascent transcripts and those that play a role in release of full-length transcripts.

Below is a description of the primary players and the events that lead to regulation of gene expression. Other components involved in gene expression, such as mRNA elongation, processing, termination, or nuclear export, can also be targeted.

Activators: Positive regulation (stimulation) of gene expression requires factors called transcriptional activators. An economical ‘recruitment’ model posits that activator proteins bind to DNA and recruit the transcriptional machinery to the promoter of the gene, thereby stimulating gene expression. Most activators are comprised of three functional modules. Of these, specificity in targeting genes is achieved by the DNA recognition module which binds to cognate DNA sequences near a promoter of a gene and in most cases DNA binding specificity is further enhanced by dimerization. A key functional module, the activating region, is thought to interact, protein-to-protein, with one or more components of the transcriptional machinery. While not wishing to be bound to a particular theory, it is believed that weak protein-protein interactions between an activating region and several components of the transcriptional machinery result in high avidity “multi-dentate” binding. In addition, the typical activating region (e.g., those used here) is also believed to contact and recruit nucleosome modifying activities to promoters.

Repressors: These proteins appear to function to inhibit gene expression at several levels. Some repressors function in part by blocking the activity of activators directly, for example, by binding to an activation domain on an activating protein in order to prevent its interaction with a component of the transcriptional machinery. Another example includes MDM-2, which not only binds to the activating region of p53, but also indirectly attenuates transcriptional activity by stimulating p53's degradation via a proteolytic pathway. More recently it has been proposed that repressors are recruited to promoters where they serve to inhibit the ability of transcriptional machinery to utilize the proximal promoter by either directly interacting with the machinery and inactivating it, or indirectly by mediating changes in chromatin structure so as to prevent the components of a transcriptional apparatus from interacting with DNA.

Transcriptional Machinery: The general components of the eukaryotic transcription apparatus have been described [Orphanides, G. et al. (1996) Genes Dev. 10, 2657-2683; Conaway. R. C. & Conaway, J. W. (1993) Annu. Rev. Biochem. 62, 161-190]. Briefly, the transcriptional machinery for mRNA comprises the catalytic core RNA polymerase II (12 subunits), several general transcription factors (TF_{II}-A, B, D, E, F, H), mediator complex (˜20 Srb and Med subunits), elongator complex, co-activator proteins and several additional polypeptides, some of which remain to be defined. Most of the these proteins are conserved through evolution and occur in species from plants to yeast to humans.

Many of the components of the transcription machinery exist in large multi-subunit complexes which associate with the RNA polymerase II, and are known as the RNA polymerase II holoenzyme. The RNA-polymerase II holoenzyme can be broadly thought to consist of two functional parts. One part is the “catalytic core” that is required for synthesizing mRNA while the other is the mediator [Bjorkland, S. & Kim, Y. J. (1996) Trends Biochem. Sci. 21, 335-337], a complex of approximately twenty proteins that is required for the holoenzyme to respond to activators. It is believed that the holoenzyme, along with additional factors that do not associate tightly (such as TBP/TFIID and a class of proteins known as co-activators [Thompson, C. M., et al. (1993) Cell 73, 1361-1375; Koleske, A. J. & Young, R. A. (1994) Nature 368, 466-469], constitutes the minimal transcriptional machinery that is recruited by activators to most promoters in vivo. Conversely, as described above, repressors function to inhibit holoenzyme activity, and in some instances they recruit co-repressor proteins.

TFIID [Burley, S. K. & Roeder, R. G. (1996) Ann. Rev. Biochem 65, 769-799], an essential component of the transcriptional machinery, is not typically found associated with the holoenzyme, and is a target of activators and some repressors as well. It is a protein complex containing about thirteen components, including TBP[Kim, J. L. et al (1993) Nature 365, 520-527; Kim, Y. et al (1993) Nature 365, 512-520] and TBP-associated factors (TAFs) [Dynlacht, B. D. et al. (1991) Cell 66, 563-576]. TBP is a sequence-specific DNA-binding protein that recognizes and binds via the minor groove to a sequence known as the TATA box (consensus: 5′-TATAAAA-3′) that exists in the promoters of many genes [Hoopes, B. C. et al. (1992) J. Biol. Chem. 267, 11539-1154; Coleman, R. A. & Pugh, B. F. (1995) J. Biol. Chem. 270, 13850-13859]. TFIID associates with TFIIA, which is comprised of three polypeptides. TFIIA helps TFIID bind to DNA perhaps by competing with repressors as well as displacing inhibitory domains within TAFs away from TBP [Geiger, J. H. et al. (1996) Science 272, 830-836; Thompson, C. M., et al. (1993) Cell 73, 1361-1375]. TFIIB, a holoenzyme component, also interacts with the promoter DNA and binds to TBP [Nikolov, D. B., et al. & Burley, S. K. (1995) Nature 377, 119-128; Burley, S. K. (1996) Nature 381, 112-113] and it is proposed to hold the entire complex together as a single unit.

Chromatin Remodeling Machinery: In order for a gene sequestered in chromatin to become available for transcription, the chromatin structure must be remodeled [Felsenfeld, G. (1992) Nature 355, 219-224; Kingston, R. E. et al. (1996) Genes Dev. 10, 905-92; Kadonaga, J. T. (1998) Cell 92, 307-313]. Chromatin remodeling occurs through activator-mediated recruitment of at least two types of chromatin remodeling complexes. The first-comprises the histone acetyl transferases that contain proteins that acetylate certain lysine residues in the amino-terminal tails of histone proteins [Brownell, J. E. & Allis, C. D. (1996) Curr. Opin. Genet Dev. 6, 176-184], thereby rendering DNA in a nucleosome more accessible to DNA-binding transcription factors. The second type of chromatin remodeling complex, Swi/Snf, uses energy derived from ATP hydrolysis to facilitate binding of the transcriptional machinery to a particular promoter [Burns, L. G. & Peterson, C. L. (1997) Mol. Cell. Biol. 17, 4811-4819; Quinn, J., et al. (1996) Nature 379, 844-847; Kwon, J., et al. (1994) Nature 370, 477-481; Cote, et al. (1994) Science 265, 65-68]. Activators can recruit chromatin remodeling complexes through direct binding. The viral activator VP16 has been shown to bind to components of both the multi-protein histone acetyl transferase (HAT) complex [Berger, S. L., et al. & Guarente, L. (1992) Cell 70, 251-265; Candan, R., et al. (1997) EMBO J. 16, 555-565], as well as the Swi/Snf complex. In fact, TFIID, another target of VP16, was observed to display a weak HAT activity [Mizzen, C. A., et al., & Allis, C. D. (1996) Cell 87, 1261-1270; Wilson, C. J., et al. & Roung, R. A. (1996) Cell 84, 235-244].

As a corollary it has been shown that certain gene-specific transcriptional repressors mediate their repressive function by recruiting histone deacetylase complexes to a target promoter [Brehm, A, et al. (1998) Nature 391, 597-601; Magnaghi-Jaulin, L., et al. & Harel-Bellan, A. (1998) Nature 391, 601-605]. Other repressors appear to directly bind histones and/or other similar proteins and these interactions lead to compact chromatin structures which occlude the transcriptional machinery.

The Regulatory Process: Based on current understanding, upon receipt of a signal, an activator bound to a promoter or enhancer recruits the chromatin remodeling machinery to the adjacent promoter. It then recruits the transcriptional machinery to form a pre-initiation complex at the promoter. It appears that assembly of a pre-initiation complex may require two synchronized steps: TFIID/TBP -TATA binding in concert with the association of the holoenzyme with the complex at the promoter [Stargell, L. A. & Struhl, K. (1996) Trends Genet. 12, 311-315]. For mRNA synthesis to be initiated at a particular gene, the complex must open (melt) the double helix to expose the template strands. Once mRNA initiation occurs and after a certain length of transcript is synthesized, the polymerase must move away from the promoter to continue mRNA synthesis. Certain activators such as HSF and Tat function to stimulate this stage of transcription process, possibly by recruiting the pTEFB complex which contains a kinase (Cdk9) capable of phosphorylating the largest of the 12 subunits of the polymerase. It has been reported that promoter escape appears to involve hyperphosphorylation of the carboxy-terminal domain of the largest subunit of the RNA polymerase II. This hyperphosphorylation achieves two goals: first, it may provide the signal to detach the mediator complex from the catalytic core; and second, it may permit the association of RNA processing and elongator complexes with the rapidly elongating polymerase.

The release of the mediator and TFIID during promoter escape by the polymerase would provide a mechanistic basis for a re-initiation event by another polymerase catalytic core [Svejstrup, J. Q., et al. & Kornberg, R. D. (1997) Proc. Natl Acad. Sci. USA 94, 6075-6078; Zawel, L., et al. (1995) Genes Dev. 9, 1479-1490]. It has been found that mediator complexes are limiting, whereas the catalytic machinery is more abundant. Moreover, activators directly interact with both the mediator as well as TBP/TFIID; thus, they may play a major role in helping to retain the mediator and/or TFIID at the promoter. Therefore, the next transcription complex can be reassembled rapidly by only recruiting the core fragment of the RNA polymerase II holoenzyme. It is postulated that re-initiation is much more likely than initiation alone to contribute significantly to rapid stimulation of gene expression. Also, activators must clearly play a role in [Ho, S. N. et al. (1996) Nature 382, 822-826] facilitating multiple rounds of transcription re-initiation.

Repression, on the other hand, requires the opposite series of events. A repressor may first directly engage an activator and mask its activating surface thereby preventing its interactions with the transcriptional and chromatin remodeling machinery. As in the case of MDM-2, after masking the activating region the repressor may also directly interrupt the low-level activator-independent assembly of the transcriptional machinery at the exposed promoter. In the next set of events, the repressor such as Retinoblastoma gene product (Rb) may directly recruit histone deacetylases, which then strip the acetyl groups off the lysine residues on histone tails. It is now believed that deacetylated histone H3 tails are then methylated by methyl transferases, which are also recruited by repressors. The methylated histone tails bind to chromatin compacting proteins such as HP-1. Thus, in a sequential manner the gene is silenced. Additional regulatory sequences that participate in stimulation as well as repression of a gene will no doubt be discovered in the future, and they also may be employed in the practice of this invention.

Compound Testing

Testing of compounds according to the invention can be conducted as follows. First, the desired compound(s) to be tested is obtained, for example, by purchase or synthesis, for example, by solid state or solution phase synthesis or recombinant techniques, as the case may be. The particular compound is typically tested in an in vitro format. For example, samples at one or more concentrations of one or more compounds (including compounds in mixtures of two or more compounds) are exposed to a cultured cell population. After exposure for a period of time appropriate for such compound to have an effect on a cell, nuclear extracts are prepared from the cells by methods well known to those of skill in the art. The nuclear extracts are then combined with a nucleic acid molecule, preferably an oligonucleotide, even more preferably a library of oligonucleotides, to allow formation of transcription factor/cis site complexes between components of the nuclear extract and cis-binding sites present in the oligonucleotides. This reaction is preferably performed under conditions that favor formation of specific transcription factor/cis site complexes to approximate those in the cells from which the extract was obtained. The profile of transcription factor binding activities is determined for each cell population, both cells exposed to the compound and cells that were not exposed to the compound. Preferably, the profiles comprise a complete profile (i.e., the pattern of all active binding activities altered by the cell's contact with the test compound). Alternatively, such profiles can comprise less than a complete profile of all changes in binding activities existing in a cell where the pattern obtained is sufficient to provide useful information such as for example, regarding the efficacy, resistance, mechanism of action and/or toxicity. As previously stated, the profiles obtained following treatment of a cell with a compound is then compared with the profile of an untreated cell to determine those transcription factor binding activities that are different between the treated and untreated cell populations. These differences indicate the biological effect(s) of exposure to the compound. Particular transcription factor binding activities can be associated with specific molecular and/or cellular effects such as apoptosis or proliferation, or practically any process that can be followed in the cells. For example, an increase in AP-1 binding activity is associated with cell activation. Further, certain binding activities as well as their relative levels can be informative as to which genes are being expressed in the cell populations involved. This information can be used to assess a variety of effects of compounds on cells, including efficacy, mechanism of action, and toxicity of compounds to which the cells had been exposed.

Preferably, the screening assays of the invention are conducted in a high throughput format, meaning that more than about 10, preferably more than about 100, 1,000, or 10,000 compounds are tested at once. The format may include an array, where either specific detection molecules or combinations thereof are located in specific locations, such as microtiter plates, slides, gels, columns, microarrays, tubes or chips. For example, arrays or other solid supports may contain detection elements for transcription factor/cis site complexes, such as antibodies that bind to proteins associated with transcription or chromatin structures, or nucleic acid molecules that hybridize to cis-binding sites. Preferably, such methods are performed where the complexes are formed and/or detected in solution, on solid surfaces, on solid supports, in semi-solid media, in gels, in column matrices, in polymer formulations, in aqueous formulations, in organic solutions, or in nonorganic solutions. High throughput formats are also often partially or fully automated.

Cells that may be used to test compounds include animal cells, plant cells, fungal cells, Archaea cells, and bacterial cells. Preferred animal cells include avian, bovine, canine, equine, feline, fish, human, murine, ovine, porcine, and primate cells. Such cells may be obtained from in vivo or in vitro (including ex vivo) sources, may be normal, diseased, transformed, infected with a virus, pathogen or other exogenous organism, or represent a particular stage of development. Cells may further include fibroblasts, epithelial, hematopoietic, CNS-derived, bone-derived, myocytes, stem cells, basal cells, and the like.

In certain embodiments, cells to be tested with a compound may be in any state of metabolism or under any physiologic condition. For example, in one aspect, cells may be treated with one or more compounds that affect the cell's metabolism or viability. Such compounds may be administered at one or more concentrations. The cells may also be pre-treated with other molecules prior to adding the particular compound of interest. Alternatively, other compounds may be added after the cells are exposed to the compound(s). Following the addition of such compounds, the cells of interest are tested for changes in their transcription factor binding activities.

In certain embodiments, the methods of the invention employ assays that use libraries of nucleic acids, e.g., oligonucleotides containing fragments representing genomic DNA, comprising one or more cis-binding sites. Initially, cells are treated (in vitro or in vivo) with one or more compounds, at one or more concentrations. The cells may also be pre-treated with other molecules prior to adding the particular compound of interest. Alternatively, other compounds may be added after the cells are exposed to the compound(s), and/or environmental conditions under which the cells are grown may be changed. In some embodiments, the cells are grown in the presence of a labeled substrate that can be incorporated into a protein. For example, a radioactively labeled amino acid can be used. Other variations of this sort will be apparent to those skilled in the art upon reading this specification.

In preferred embodiments, the methods of the invention employ libraries of nucleic acid molecules. In another embodiment, the library may comprise a population of nucleic acid molecules containing known binding sites for transcription factors. In still other preferred embodiments, the nucleic acid molecules used in the methods according to the invention will each contain at least one binding site. In certain embodiments, the oligonucleotides comprise 2, 3, 4, 5, 6, 7, 8, 9, or 10 binding sites. Each nucleic acid molecule may contain a different binding site or some binding sites may be in common among multiple nucleic acid molecules. Such nucleic acid molecules may comprise defined nucleic acid sequences. Certain preferred nucleic acid molecules comprise nucleic acid sequences that are representative of a genome. Other preferred nucleic acid molecules comprise nucleotide sequences found in genomic DNA. Alternatively, the nucleic acid sequence may be random. A “defined nucleic acid sequence” refers to a specific sequence of nucleotides, and is typically represented in the 5′ to 3′ direction using standard single letter notation, where “A” represents adenine, “G” represents guanine, “T” represents thymine, and “C” represents cytosine. It will be appreciated that a nucleic acid molecule having a defined nucleotide sequence may include a different nucleotide at the same position, ie., is degenerate at that position, with respect to one or more positions in the particular sequence. Degenerate bases may be represented by any suitable nomenclature, for example, that which is described in World Intellectual Property Organization Standard ST.25 (1998), Appendix 2. Random nucleic acid molecules may also comprise nucleotide sequences representative of a genome. For example, a nucleic acid molecule may comprise the same bias for nucleotide representation as a particular genome.

Nucleic acid molecules may be synthetic or isolated from cells, varying in length from about 4 to about 1000 nucleotides in length, comprise purified DNA, partially-purified DNA or unpurified DNA, and may comprise DNA within chromatin, a chromosome, or chromosome segment. Oligonucleotides may be representative of or a part of a genome comprising human, mammalian, vertebrate, animal, plant, fungi, eukaryotic, prokaryotic or viral genomes. Nucleic acid molecules may contain modified nucleotides, for example, methylated nucleotides, as well as, or alternatively, nucleotide analogs and derivatives. Nucleic acid molecules may also comprise a first amplification primer site upstream of the transcription factor binding site and a second amplification primer site downstream of the binding site.

In one embodiment, a random DNA library is generated for use in the binding reactions with nuclear or other proteins. For this, a mixture of oligonucleotides, each with a fully randomized central domain flanked by two fixed but different sequences on either side, is synthesized. The fixed sequences are typically at least 15 nucleotides long to allow for efficient primer annealing, while the randomized sequence is typically at least 10 nucleotides in length. Using a primer complementary to the fixed region at the 3′ end of each oligonucleotide in the library and another primer complementary to the fixed region at the 5′ end of each oligonucleotide, the double-stranded random DNA library is generated by at least one and up to five cycles of PCR.

In another embodiment, a genomic DNA library containing oligonucleotides representative of human genomic DNA is used. The library can be generated by a method similar to the one described by Singer et al. (1997, Nucleic Acids Res. 25, 781-786). A primer consisting of a fixed 5′-region (18-22 bp in length) and a 9-nucleotide randomized extension at its 3′-end is annealed to denatured genomic DNA and extended with Klenow DNA polymerase. Extension products are isolated and the process is repeated with a second primer having a different fixed region. The DNA is purified and further amplified by PCR using primers containing only the fixed sequences. The amplified DNA is size-fractionated using polyacrylamide gel electrophoresis and then amplified again with primers A and B and gel-purified again to yield genomic libraries containing inserts of defined size ranges. A genomic library prepared in this way consists of double-stranded DNA molecules that each contain a genomic DNA sequence in the center (typically 25-250 bp in length) flanked by two different fixed regions (priming sequences) at either end. Preferred genomic libraries contain 35-100 bp of center DNA, and even more preferred are genomic libraries containing 45-50 bp of center DNA.

Nuclear extracts containing nuclear proteins, for example, activators, repressors, transcription factors, and proteins involved in chromatin structure formation, maintenance, and/or remodeling, are obtained from the cells exposed to the compound. Nuclear extracts can be prepared by any suitable method, including by hypotonic lysis on ice, pelleting of nuclei and extraction of proteins in high salt buffer, and then dialysis or dilution to 100 mM salt, storage at −80° C. Nuclear extracts may be obtained at a single time point following exposure to the compound, or at different times. Extracts may also be obtained from cells treated at varying concentrations of a compound or with mixtures of more than one compound. These nuclear extracts will exhibit changes in protein composition and concentration according to the type of compound as well as concentration and duration of treatment. It is these changes in the DNA binding proteins or transcription factors in treated cells compared to untreated cells, or among cells treated according to different treatment regimens, that cause changes in binding activities that can be profiled and used to determine effects of the compounds on cells.

The nuclear extracts are combined with the DNA library to generate the binding reaction, which typically contains 5-10 μg of nuclear extract proteins (or various protein fractions or other protein preparations), 5-50 ng of double-stranded library DNA (see above), and non-specific competitor nucleic acids such as polydI:dC, salmon sperm DNA, calf thymus DNA, or E.coli total RNA. One strand of the library DNA may be biotinylated at its 5′-end, or otherwise modified such that purification from the binding reactions can be carried out using solid phase chemistries. The salt and buffer conditions are typically 1-5 mM MgCl₂, 50-100 mM KCl, 20-25 mM HEPES-NaOH or Tris-HCl (pH 7.5-8.0), 10-20% glycerol, 0.1 mM EDTA. Incubation temperature and time are typically 4 C. or room temperature and between 15 minutes and 3 hours, respectively.

After sufficient time for binding, in an assay wherein the nucleic acids are in solution, the bound protein/DNA complexes can be partitioned away from unbound components using properties such as molecular weight, charge, or other physical or chemical properties. One preferred embodiment involves using the electrophoretic mobility shift assay (EMSA) which allows separating large numbers of bound complexes from unbound nucleic acids and/or binding factors. The recovered complexes can then be isolated and the individual nucleic acid and protein components further purified for direct analysis if desired. For example, it is well known in the art that nucleic acids can be purified from the isolated protein/DNA complexes by one of several methods. The sample containing the protein/DNA complexes can be extracted by organic solvents (phenol, chloroform) and the nucleic acids can be precipitated by the addition of 2-3 volumes of ethanol and recovered by centrifugation. Alternatively, when using biotinylated DNA, nucleic acids can be captured with streptavidin-coated agarose beads, also making use of magnetic separation. Chemical methods for attaching the detectable label biotin (i.e., biotinylating) are known in the art. See, e.g. Agrawal, Chapter 3 in Protocols For Oligonucleotide Conjugates, Volume 26, Humana Press, Totowa, N.J. 1994, pages 93-120 (see especially pages 108-109) and Chu et al., Chapter 5, Id., pages 145-165 (see especially page 157). Oligonucleotides and other nucleic acids can also be biotinylated using enzymatic systems such as, e.g., nick translation (E. coli DNA Polymerase I and Dnase I; Boyle, Section V of Chapter 3, in Short Protocols in Molecular Biology, Second Edition, Ausubel, et al. Editors John Wiley & Sons, New York, 1992 pages 3-41 to 3-44) or “tailing” reactions using terminal deoxynucleotidyl transferase (see, e.g., the LABEL-IT™ 3′ Biotin End Labeling Kit from CPG, Inc., Lincol Park, N.J.). Other methods of DNA capture include nucleic acid hybridization and solid phase chemistries. Proteins can be purified from the isolated protein/DNA complexes by dissociation from the DNA in the presence of an ionic detergent (e.g., sodium dodecyl sulfate), concentrated by filtration, or precipitated by the addition of high concentrations (2-4 M) of ammonium sulfate.

The eluted DNA fragments, if captured using streptavidin-coated beads, is then recovered from the beads using standard techniques known to those in the field and appropriate to the type of bead. The DNA fraction, which represents the “protein bound” fraction of the original library, can be amplified by PCR or another nucleic acid amplification method to a moderate level and then used in a binding reaction identical to the first reaction.

The binding process can be repeated any number of rounds, depending upon the level of selectivity desired. Typically 2 or 3 rounds are sufficient to achieve a significant selection of transcription factor binding activities and a negligible level of background. The DNA fraction can also be analyzed directly without amplification.

The isolated nucleic acid fragments can be analyzed for the presence of transcription factor binding sites and for the level of transcription factor binding activities using a number of methods, including direct DNA sequencing and hybridization techniques. With direct sequencing, the individual oligonucleotides selected in the binding reactions are sequenced and the transcription factor binding sites are identified and counted on the selected oligonucleotides. For hybridization, the isolated fragments could be labeled in a way that would allow detection (e.g., by radioactivity, biotin-avidin, ezymatically) and then hybridized to a membrane or array that contains single-stranded DNA oligonucleotides specific for particular cis-binding sites. In this embodiment, the nucleic acid fragments could be hybridized to a nucleic acid array comprising a plurality of binding site-specific oligonucleotides, wherein hybridization could be detected using a variety of methods well known in the art.

In contrast, when the nucleic acids are bound to a solid support, e.g., as a nucleic acid array, labeled proteins that interact therewith can be detected. Those in the art will also appreciate that an unlabeled transcription factor bound to its cognate cis-binding site can also be detected in other ways, for example, using detectable antibodies or other epitope-specific moieties.

The results of these assays are then compared with the results of one or more control assays. In certain preferred embodiments, the control assay concerns obtaining a nuclear extract from cells that have not been exposed to the compound, or which have been exposed to the compound under different conditions, for example, at different concentrations, for differing periods of time, etc. Differences in results reveal which transcription factor binding activities are affected by the compound, which can be used as an indicator of biological effects for that particular compound.

Because many particular transcription factor binding activities are involved, for example, with regulating the expression of some, but not all genes, further studies can be undertaken to investigate the compound-mediated effects on expression of such genes. Once a particular transcription factor binding activitiy is identified, the genes with which it is functionally associated (i.e., those genes over which it has some regulatory influence, be it activation, repression, sequestering in chromatin, etc.) can be determined. This determination can be made, for example, by searching sequence databases to determine which genes the relevant cis-binding site is proximal to in the genome. If desirable, these results can be confirmed experimentally. A database of genes whose expression is at least partially controlled by particular transcription factors and/or cis-binding sites can be established. Carried to its conclusion, a database of all regulatory elements and the genes whose expression they control can be developed.

From such information, some or all of the genes whose expression may be influenced by a particular transcription factor can be identified. Accordingly, a nucleic acid array containing hybridization probes specific for some or all of the genes functionally associated with the particular transcription factor (or set of particular transcription factors) can be prepared. Messenger RNA from cells treated with a compound known to influence the particular transcription factor binding activity can be exposed to the array, and changes in the expression of specific genes can be assessed by such RNA profiling. As will be appreciated, frequently not all genes whose expression is at least partially controlled by a particular transcription factor will be expressed in a particular cell, given that expression often requires coordination between multiple factors.

An approach similar to RNA profiling detects and quantifies the transcription factor proteins, as the proteins encoded by particular genes can also be readily determined and detected. These proteins can be over-expressed in appropriate expressions systems as are understood by those of skill in the art and, for example, high affinity polyclonal, and preferably monoclonal antibodies, raised against each of them. Such antibodies can be arrayed on a solid support in a manner analogous to different nucleic acid hybridization probes. Cell extracts from treated and untreated cell populations can be used in binding reactions to form the transcription factor/cis site complexes characteristic of each of those cell populations. To characterize the complexes from each of the cell populations, they can be added to such antibody arrays and the level of each transcription factor determined. The results of such binding may be detected by any suitable technique, for example, by using a second, labeled antibody specific for a different epitope on the transcription factor so as to create a probe antibody-protein-detection antibody sandwich. This allows the profiling of which particular transcription factor binding activities are present in cells exposed to the compound compared to cells that were untreated.

Another exemplary technique that can be used in the practice of the invention involves contacting oligonucleotides in a nucleic acid library with transcription factors obtained from nuclear extracts of cells treated with a compound, allowing the factors to form transcription factor/cis site complexes with specific oligonucleotides of the library, and separating the complexes from free constituents of the reaction using electrophoretic mobility shift assays (EMSA). Meaningful data is derived by comparing EMSA results for extracts from cells treated and not treated with the compound, or by comparing EMSA results for cells treated with the compound for different periods of time, at different concentrations, or in the presence of other compounds.

Determining Efficacy of Compounds in Cells

Cells can be treated with various compounds developed to exert particular effects on cells, e.g., inhibition of growth, inhibition of particular enzymes or other gene products, and production of particular gene products (among many others). In addition to specific assays for the desired effect, e.g., production of a particular gene product, the effect of the compound can be determined by studying changes in gene expression. This is accomplished by first determining the transcription factor binding activities for both the treated and untreated cell populations and obtaining a binding activity profile for each population. Secondly, the profiles are compared to each other to determine which binding activities change as a result of the compound treatment. Certain binding activities, as well as the relative levels of these activities, can be informative as to which genes are being expressed in the cell populations involved. Preferably, in the same assay, various concentrations of the compound (or mixture) are tested. Also, such determinations can be conducted at various time points after exposure to the compound(s). Preferably, the assay is implemented in a high throughput, preferably automated, format.

To perform the binding assays to examine the effects of such compounds on gene expression, a nuclear extract from each treated cell population, as well as from untreated cells, is prepared. The profile of transcription factor binding activities identified as having been affected can be used to assess efficacy of the particular compound.

Binding activities that become activated or, alternatively, that are repressed in response to compound treatment provide information as to which genes, or subset of genes, are activated or repressed, as the case may be, in response to exposure to the compound. Additional studies on one or more of these genes can then be carried out. For example, a nucleic acid array comprising genes known to be regulated by the particular transcription factor can be used to perform RNA expression profiling to further understand the effect of the compound on particular gene(s).

Determining Toxicity of Compounds on Cells

The methods of the invention can also be used to assess toxicity or other adverse effects of various compounds on cells. A preferred method useful in performing such assays is carried out on cells treated with one or more particular compounds at various concentrations. The assay is performed on cells at various time points after exposure to the compound(s) on a prepared nuclear extract from each treated cell population, as well as from untreated cells. The effect of the compound is determined by first defining the transcription factor binding activities for both the treated cell population and for the untreated cell population. These profiles of binding activities are comprised of both the types of binding activities present in the cells as well as how active they are relative to each other. The profiles are then compared to each other to determine those transcription factor binding activities that are different between the treated and untreated cell populations and thus a result of treatment with the compound. Changes in particular binding activities are indicative of certain molecular and cellular changes in the cells. Thus, the profiles involving transcription factors and their cognate binding sites that are activated or repressed in the treated versus untreated cells and that correlate with toxicity allow toxicity of the particular compound to be assessed. Additional studies on one or more of these transcription factor binding activities shown to be altered can then be carried out. For example, a nucleic acid array comprising genes known to be regulated by the particular transcription factor can be used to perform RNA expression profiling to further understand the mechanism of toxicity of the compound.

Such compounds, originally discovered or designed to exert specific benefits such as therapeutic effects, can be studied further for their effects on gene regulatory elements. Changes in activity of certain regulatory elements may be predictive or otherwise informative regarding extent and mechanism of adverse effects.

Determining Mechanism of Action of Compounds in Cells

The profile of active or silenced gene regulatory elements in cells treated with a particular compound can also give important information concerning mechanism of action. Changes in activity of particular transcription factor binding activities can denote changes in expression of certain genes, which can also be further studied using additional experimental approaches such as RNA expression profiling.

In this application, assays can be carried out on cells treated with a particular compound at various concentrations and at various time points. The starting cells may also be varied, e.g., at various levels of confluency, synchronized with regard to cell growth, or serum-starved, before treatment. A nuclear extract from each treated cell population, as well as from untreated cells, is prepared and the profile of transcription factor binding activities that result from exposure to the compound are determined and compared according to the embodiments of the invention in order to determine the effect on transcription factor binding activity. Effects of the compound on the expression of particular genes, particularly those regulated by specific transcription factors, can then be assessed.

Optimizing Lead Compounds

The methods of the invention can also be used to correlate the structure/function relationship of families of compounds or particular moieties with activity of specific regulatory elements. Changes in activity of particular transcription factor binding activities, as well as the genes they regulate, can be used as a measure of potential beneficial activity as well as undesired side effects. Assays are carried out on cells treated with the various families or classes of compounds, preferably at various concentrations and at various time points. The profile of transcription factor binding activities after treatment with each compound is determined and compared in order to help determine the optimal compound(s) for each desired effect. Effects of the various compounds on the expression of particular genes regulated by specific transcription factors of interest can then be assessed, e.g., by RNA expression profiling. This process can be used in an iterative fashion to obtain a compound, or class of compounds, having the desired activity, but having few if any undesired effects on gene transcription. Such methods allow rapid progress to be made with regard to initial lead compound identification and subsequent lead optimization.

Compounds

The methods of invention contemplate detecting changes in transcription factor binding activities reflective of gene expression changes induced directly or indirectly by any compound, including but not limited to: proteins, peptides, nucleic acids, lipids, carbohydrates, organic or inorganic molecules, hormones, small molecules, polymers etc. Such compounds can be naturally occurring macromolecules, or synthetic derivatives, analogs or mimetics of these macromolecules. Such a broad array of compounds, when in contact with cells, will affect transcription factor binding activities differently, so that when the profiles between cells treated with the various compounds or under various conditions are compared according to the invention

In order to synthesize a compound for testing in the first instance, any suitable method may be employed. Such methods include the synthesis of a single compound by traditional methods, up through a massively parallel combinatorial approach. For example, a number of combinatorial synthetic methods are known in the art. For example, Thompson & Elman ((1996), Chem. Rev., vol. 96, 555) recognized at least five different general approaches for preparing combinatorial libraries on solid supports. These were: (1) synthesis of discrete compounds; (2) split synthesis (split and pool); (3) soluble library deconvolution; (4) structural determination by analytical methods; and (5) encoding strategies in which the chemical compositions of active candidate are determined by unique labels, after testing positive for biological activity. Synthesis in libraries in solution includes at least spatially separate synthesis, and synthesis pools. Additional descriptions of combinatorial methods are known in the art. See, e.g., Lam, et al. (1997) Chem. Rev., vol. 97; 4111.

These approaches can be readily adapted to prepare compounds for use in accordance with the methods of the present invention, including suitable protection schemes, as necessary. Synthesis and testing of the various compounds in accordance with the instant methods allow a wide range of compounds to be tested. Such compounds can then be subjected to further study, for example, by RNA profiling. In addition, they can serve as lead compounds for optimization by medicinal chemistry or other programs.

The invention now will be discussed with reference to particular preferred embodiments, which, for convenience, will be in the context of oligonucleotides, but it is to be understood that the invention is not limited to such context and may be applicable to other nucleic acid, e.g., genomic nucleic acids.

The following examples are provided to assist in understanding the present invention. The examples and experiments described below should not, of course, be construed as specifically limiting the invention and such variations of the invention, now known or later developed, which would be within the purview of one skilled in the art in view of the description provided herein.

### EXAMPLE 1

Isolation of Transcription Factor/Cis Site Complexes

A. Cell Growth, Treatment with Compound, and Harvest

Jurkat cells were grown in RPMI 1640 medium supplemented with 10% fetal bovine serum, antibiotics/antimyotics, 1% L-Glutamine, and 1% non-essential amino acids (Gibco BRL). At a cell density of 1-5×10⁶ cells/ml, cells were split into two equal aliquots. One aliquot was treated with a combination of 100 ng/ml Phorbol 12-myristate 13-acetate (PMA) and 2 ug/ml Ionomycin in DMSO (Activated Jurkats) and the other aliquot was treated with DMSO alone (Resting Jurkats), both for 2-3 hrs. Following treatment, cells were transferred to centrifuge tubes and pelleted by brief centrifugation at room temperature, then washed with 5 ml ice cold phosphate-buffered saline. Cell pellets were then placed on ice and processed to isolate nuclear proteins (see Nuclear Extraction, below).

B. Nuclear Protein Extraction

Extraction of nuclear proteins was performed according to published procedures [Skerka, C., et al (1995) J. Biol. Chem. 270, 22500-22506; Andrews, N. C. & Faller, D. V. (1991) Nucl. Acids Res. 19:2499; Dignam, J. D., et al. (1983) Nucl. Acids Res. 11:1475-1489], with minor modifications. Unless otherwise specified, all reagent manipulations were performed on ice and all centrifigations were at room temperature. Cell pellets were resuspended in 250-500 ul hypotonic lysis buffer (10 mM HEPES, pH 7.9, 10 mM KCl, 1.5 mM MgCl₂, 1 mM EDTA, pH 8.0, 10% glycerol, 0.5 mM DTT, 0.15% NP-40, 1 mM PMSF) by vigorously vortexing, and incubated on ice for 10 minutes. Nuclei were pelleted by centrifugation at 1,000×g for 7 minutes, the supernatant was decanted, and the nuclei were resuspended in nuclear extraction buffer (10 mM HEPES, pH 7.9, 420 mM NaCl, 1.5 mM MgCl₂, 1 mM EDTA, pH 8.0, 10% glycerol, 0.5 mM DTT, 1 mM PMSF) by vigorous vortexing. Samples were incubated on ice for 30 minutes and then centrifuged for 5 minutes to pellet extracted nuclei. Supernatant (Nuclear Extract) was transferred to a fresh tube and either immediately flash frozen or dialyzed against Nuclear Extraction Buffer containing 150 mM NaCl prior to freezing. A small aliquot of the Nuclear Extract was removed prior to freezing to quantify protein concentration using the Bradford assay, a procedure well known in the art.

Successful stimulation of Jurkat cells by PMA/ionomycin treatment was examined by performing EMSA with control and activated nuclear extracts and short DNA oligonucleotides corresponding to the binding sites for Oct-1, NF-κB, and AP-1, as described below.

C. DNA “Bait” Library Preparation

The genomic DNA library was generated by a method similar to the one described by Singer et al. (1997, Nucleic Acids Res. 25, 781-786). A primer consisting of a fixed 5′-region (18-22 bp in length) and a 9-nucleotide randomized extension at its 3′-end was annealed to denatured genomic DNA and extended with Klenow DNA polymerase. Extension products were isolated and the process was repeated with a second primer having a different fixed region. The DNA was purified and further amplified by PCR using primers containing only the fixed sequences. The amplified DNA was electrophoresed on a native polyacrylamide gel and various size-ranges of DNA were cut out and eluted from the gel (e.g. 75-100 bp, 100-125 bp, 125-150 bp, etc.). The gel-purified DNA was amplified again with primers A and B and gel-purified again to yield genomic libraries containing inserts of defined size ranges. A genomic library prepared in this way consisted of double-stranded DNA molecules that each contained a genomic DNA sequence in the center (typically 25-250 bp in length) flanked by two different fixed regions (priming sequences) at either end.

D. Binding Reaction

The binding reaction typically contained 5-10 μg nuclear extract proteins, 5-50 ng double-stranded library DNA (see above), and non-specific competitor nucleic acids such as polydI:dC, salmon sperm DNA, calf thymus DNA, or E.coli total RNA. One strand efthe library DNA was biotinylated at its 5′-end. The salt and buffer conditions were typically 1-5 mM MgCl₂, 50-100 mM KCl, 20-25 mM HEPES-NaOH or Tris-HCl (pH 7.5-8.0), 10-20% glycerol, 0.1 mM EDTA. Incubation temperature and time are typically 4 C. or room temperature and between 15 minutes and 3 hours, respectively.

After sufficient time for binding, the bound protein/DNA complexes were partitioned away from unbound components by electrophoretic mobility shift assay (EMSA). The eluted DNA fragments were captured using streptavidin-coated beads and then recovered from the beads, using methods appropriate to the type of bead. The DNA fraction, which represents the “protein bound” fraction of the original library, was amplified by PCR to a moderate level and then used in binding reaction identical to the first reaction.

A total of three rounds of binding were completed, a representative fraction of the selected DNA fragments was directly sequenced, and the sequences were then examined for the presence of known transcription factor cis-binding sites.

E. Determining Efficacy of Compounds in Cells

As an example of compound efficacy screening, the effects of TPA/ionomycin on T cells was tested by carrying out an assay on nuclear extracts from TPA/iomomycin-activated Jurkat cells compared to that from resting Jurkat cells. The results of such an experiment are shown in Table I.

TABLE I

| DNA data set - cis site | RESTING CELLS | ACTIVATED CELLS |
|---|---|---|
| designation | 27881 bp 100% | 29776 bp 100% |
| AHRARNT | 2.7 | 1.0 |
| AP1 | 1.9 | 10.5 |
| CAAT | 10.1 | 11.1 |
| CDP | 0.7 | 1.6 |
| CREL | 1.5 | 3.0 |
| E2F | 0.7 | 1.9 |
| GATA2 | 2.6 | 1.1 |
| HFH1 | 1.2 | 0.3 |
| HNF1 | 0.2 | 1.1 |
| ISRE | 0.2 | 1.0 |
| LDSPOLYA | 3.2 | 1.6 |
| MYCMAX | 10.1 | 10.1 |
| MYOGNF1 | 1.7 | 3.0 |
| NF1 | 3.2 | 4.1 |
| NFKAPPAB50 | 0.7 | 1.6 |
| OCT1 | 12.6 | 7.0 |
| S8 | 3.8 | 1.1 |
| SREBP1 | 6.3 | 6.2 |
| STAT3 | 0.2 | 2.1 |
| VDR-RXR2 | 0.2 | 1.3 |
| VMAF | 0.5 | 1.9 |

To generate the data of Table I, 586 genomic DNA fragments (containing a total of 27881 bp) that bound to proteins in resting Jurkat cell nuclear extract, and 631 genomic DNA fragments (29776 bp) that bound to proteins present in TPA/ionomycin-activated Jurkat cell nuclear extract were sequenced. The presence of known transcription factor binding sites in these fragments was used to form the activity profile and their activity determined by searching for the corresponding consensus motifs for those factors. A partial list of these cis-binding sites can be found in the first column of the Table. The second and third columns show the numbers of the corresponding binding sites identified by the assay using the resting and activated Jurkat nuclear extracts, respectively (expressed as the percentage of DNA fragments containing the sites). The results show that certain cis sites, e.g. AP-1, are strongly induced in activated Jurkat nuclear extracts, while others, e.g., MycMax or CAAT-box, are unchanged between the two cell populations. The observed induction of AP-1 is consistent with the known TPA/ionomycin-induced activation of many genes containing an AP-1 binding site in their promoter. It can therefore be concluded that genes containing other cis elements that show a difference in abundance in the two data sets would be regulated accordingly (i.e., STAT3 binding sites are predicted to be present in TPA/ionomycin-activated gene promoters). Thus, the method of the invention provides profile data regarding aspects of gene expression and reflecting the effect of a compound on a cell population.

### EXAMPLE 2

Determining Toxicity of Compounds on Cells

In a particular example of determining toxicity of compounds on cells, MCF7 cells were grown in high glucose DMEM containing 10% fetal calf serum, antibiotic/antimyotics, and supplemented with 2 mM L-glutamine. For drug treatment, cells were grown to a density of approximately 1×10⁶ cells/ml and an additional 10% media volume containing 1.85-18.6 ug/ml (in 95% EtoH)Tamoxifen, 0.17-8.54 ug/ml (in 95% EtoH) Taxol, or 0.56-2.9 ug/ml (in water) Doxorubicin was added and gently mixed. Cells were harvested after 2-6 hr incubation and nuclear extracts were prepared as described above. Toxicity of the drugs was monitored by treating parallel samples and assaying for cell death by Trypan Blue staining at 24 hrs, 48 hrs, and 72 hrs post treatment.

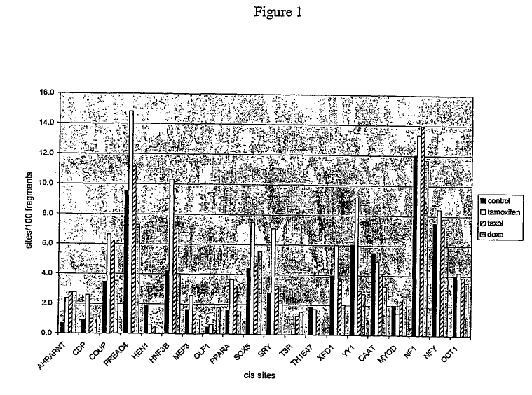

Nuclear extracts were each mixed with a library of genomic DNA fragments and fragments forming specific complexes with nuclear proteins were sequenced. For each of the four samples, about 800 fragments were sequenced and searched for the presence of cis-binding sites as described for in Example 1. The data are presented in the bar graph shown in FIG. 1. It can be seen that the percentage of nucleic acid fragments containing selected cis-binding sites that were isolated in binding assays with nuclear extract from untreated cells (black bars) varies markedly from cells treated with tamoxifen (white bars), taxol (hatched bars) or doxorubicin (gray bars). Thus, the method of the invention provides a profile of transcription factor binding activities that are useful in establishing a link between toxic effects of compounds (such as those of the example) and changes in gene expression.

### EXAMPLE 3

Determining Mechanism of Action of Compounds in Cells

In a specific example, PC12 cells were grown in high glucose DMEM containing 10% horse serum and 5% fetal calf serum. For differentiation, cells were transferred to serum-free medium containing N-2 Supplement (Life Technologies) and 200 ng/ml Nerve Growth Factor-beta (Sigma). Cells were harvested after 5 hr and nuclear extracts were prepared as described above. A positive response of the PC12 cells to NGF was monitored by EMSA of AP-1 activity.

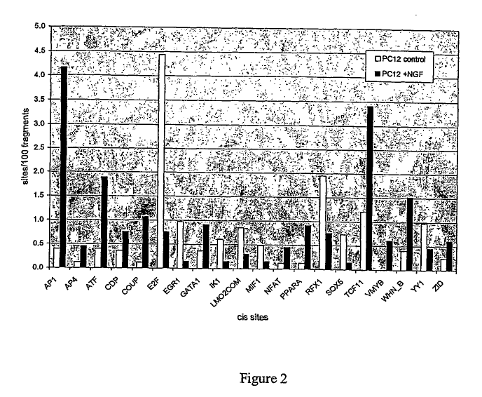

In this example, nuclear extracts were incubated with a random 16-mer library and the DNA present in specific protein/DNA complexes was isolated and sequenced. Known transcription factor binding sites present in the DNA sequences were counted as described in Example 1 and the data generated are presented in FIG. 2 in the same mainer as in Example 2. Thus, the bars indicate the percentage of DNA fragments containing selected cis sites that were isolated in binding reactions containing nuclear extracts from either untreated (white bars) or NGF-treated (black bars) PC12 cells. It can be seen that NGF-treatment leads to an increase in binding activity for AP 1, ATF, TCF11 (among others), while for example E2F and RFX1 activities are reduced after NGF treatment. Even though this profile represents only a partial analysis, it indicates that genes regulated by AP1, ATF, or TCF11 may be activated upon NGF treatment, while genes regulated by E2F and RFX1 are expected to be repressed upon NGF treatment.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention clained. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of the inventions disclosed herein.

The inventions have been described broadly and generically herein. Each of the narrower species and subgeneric groupings falling within the generic disclosure also form part of these inventions. This includes the generic description of each invention with a proviso or negative limitation removing any subject matter from the genus, regardless of whether or not the excised material is specifically recited herein.

Other embodiments are within the following claims. In addition, where features or aspects of an invention are described in terms of a Markush group, those skilled in the art will recognize that the invention is also thereby described in terms of any individual member or subgroup of members of the Markush group.

We claim:

1. A method for determining a biological effect of a compound on a transcription factor binding activity profile of a cell, the method comprising:
(a) obtaining a nuclear extract from cells exposed to said compound;
(b) combining said nuclear extract with a nucleic acid containing a cis-binding site under conditions that allow formation of transcription factor/cis site complexes; and
(c) comparing if any transcription factor/cis site complex formed as a result of step (b) differs from transcription factor/cis site complexes formed by combining the nucleic acid with a control nuclear extract obtained from cells not exposed to the compound.

2. A method according to claim 1 wherein the nucleic acid contains a plurality of different cis site.

3. A method according to claim 1 wherein the nuclear extract is combined with a plurality of nucleic acid species, wherein each nucleic acid species contains a different cis site.

4. A method according to claim 3 wherein the plurality of nucleic acid species comprises a library of oligonucleotides.

5. A method according to claim 4 wherein the oligonucleotides in the library comprise nucleotide sequences that are representative of a genome.

6. A method according to claim 4 wherein at least a portion of the nucleotide sequences of the oligonucleotides in the library are random nucleotide sequences.

7. A method according to claim 4 wherein the oligonucleotides in the library comprise at least one nucleotide that is modified.

8. A method according to claim 7 wherein the modification is methylation.

9. A method according to claim 4 wherein the oligonucleotides in the library comprise at least one nucleotide analog.

10. A method according to claim 4 wherein the oligonucleotides in the library comprise a first amplification primer site upstream of the cis site and a second amplification primer site downstream of the cis site.

11. A method according to claim 3 wherein the plurality of nucleic acid species comprises genomic DNA fragments.

12. A method according to claim 1 wherein the cell is selected from the group consisting of a vertebrate cell and a pathogen.

13. A method according to claim 1 wherein the cell is a mammalian cell selected from the group consisting of a canine, equine, feline, murine, ovine, porcine, and primate cells.

14. A method according to claim 1 wherein the cell is a human cell.

15. A method according to claim 1 wherein the cell is selected from the group consisting of a diseased cell, a normal cell, and a pathogen.

16. A method according to claim 15 wherein the cell is a diseased cell selected from the group consisting of a cancer cell, an infected cell, an abnormal T cell, and abnormal neuronal cell.

17. A method according to claim 15 wherein the cell is a pathogen selected from the group consisting of a eukaryotic cell, a prokaryotic cell, and a virus.

18. A method according to claim 1 wherein the test compound is selected from the group consisting of a small organic molecule, a lipid, a carbohydrate, a peptide, a polypeptide, a mutant polypeptide, and a nucleic acid.

19. A method according to claim 1 comprising a plurality of test compounds.

20. A method according to claim 3 wherein the plurality of nucleic acid species are attached to a solid support.

21. A method according to claim 20 wherein each species of nucleic acid within the plurality of nucleic acid species is localized at a different location on the solid support.

22. A method according to claim 1 implemented in a high throughput format.

23. A method according to claim 1 further comprising performing RNA profiles of the cells cultured in the presence or absence of the compound.

24. A method according to claim 23 wherein the RNA profile is performed using a nucleic acid array.

25. A method according to claim 24 wherein the nucleic acid array comprises hybridization tags for a subset of genes expressed in the cells.

26. A method according to claim 1 wherein proteins in the nuclear extract are labeled for detection.

27. A method according to claim 1 wherein the biological effect is selected from the group consisting of toxicity, efficacy, and mechanism of action.

28. A method for determining a biological effect of a compound on a transcription factor binding activity profile of a cell, the method comprising:
(a) obtaining a nuclear extract from cells exposed to said compound;
(b) reacting the nuclear extract with a solid support to which is attached a detection element for specific interaction with a protein associated with regulating transcription of one or more genes under conditions that allow formation of a detection element/protein complex; and
(c) comparing if any detection element/protein complex formed as a result of step (b) differs from detection element/protein complexes formed by combining the detection element with a control nuclear extract obtained from cells not exposed to the compound.

29. A method according to claim 27 wherein the detection element is a nucleic acid molecule containing a cis site.

30. A method according to claim 27 wherein the detection element is an antibody for a protein associated with regulating transcription.

31. A method for determining a biological effect of a compound on a transcription factor binding activity profile of a cell, the method comprising:
(a) obtaiing a nuclear extract from cells exposed to said compound;
(b) reacting the nuclear extract with a solid support to which is attached a detection element for specific interaction with a protein associated with chromatin structure under conditions that allow formation of a detection element/protein complex; and
(c) comparing if any detection element/protein complex formed as a result of step (b) differs from detection element/protein complexes formed by combining the detection element with a control nuclear extract obtained from cells not exposed to the compound.
